(19) United States
(12) Patent Application Publication
(10) Pub. No.: US 2007/0116645 A1
(43) Pub. Date: May 24, 2007

(54) Methods and compositions for inhibiting cholesterol uptake

(76) Inventors: Steven Farber, Baltimore, MD (US); Eric J. Smart, Versailles, KY (US)

Correspondence Address: David S Rastrick;Nixon Peabody, 100 Summer Street, Boston, MA, 02110-2131, US

(21) Appl. No.: 10/576,388

(22) PCT Filed: Feb. 3, 2004
(86) PCT No.: PCT/US04/03020

§ 371 Date: Dec. 20, 2006

(51) Int. Cl. A61K 49/00; A61K 39/395

(52) U.S. Cl. 424/9.2; 424/145.1

(57) ABSTRACT

The present invention is directed to a method for the lowering of levels of LDL cholesterol in an individual comprising administering to the individual an agent which modulates the activity of the protein annexin 2, cyclophilin A, cyclophilin 40, or HSP 56 or the complex of annexin 2 and caveolin I, in the intestinal cells of the individual.

### GOVERNMENT SUPPORT

This invention was supported by the National Institutes of Health National Heart, Lung, and Blood Institute grants HL58475, HL64056, HL62844, National Institute of Diabetes and Digestive and Kidney Diseases DK60369, and National Institute of General Medical. Sciences GM63904 and the government of the United States has certain rights thereto.

### BACKGROUND OF THE INVENTION

Hyperlipidemias, particularly hypercholesterolemia and the hyperlipoproteinemias, are among the most potent risk factors in the causation of atherosclerosis. Hyperlipoproteinemias are also implicated in the development of pancreatitis. A long-established theory suggests that the higher the circulating levels of cholesterol, usually in the form of low density lipoproteins (LDLs) containing cholesterol, the more likely it is to gain entrance to the arterial wall and cause atherosclerosis. (Brown and Goldstein, “The Hyperlipoproteinemias and Other Disorders of Lipid Metabolism,” in Harrison's Principles of Internal Medicine 1650-1661 (Braunwald et al., 1987)).

Cardiovascular disease is the leading cause of death in women and middle-aged American men. In 1988, more than 41,000 U.S. residents died of cardiovascular disease before the age of 50. Atherosclerosis, however, which is known to contribute to cardiovascular disease and stroke, begins at a much earlier age. Fatty streaks are common in the arterial walls of children, and a high prevalence of coronary-artery lesions has been found in young men who die accidentally or violently. Children and adolescents with elevated serum cholesterol levels are more likely than their counterparts with normal cholesterol levels to have parents with coronary heart disease. Higher serum cholesterol levels in childhood have been associated with aortic atherosclerosis at autopsy in adolescents and young adults, and both aortic and coronary atherosclerosis in men ranging from 15 to 34 years of age have been correlated with postmortem cholesterol levels (Klag et al., New Eng. J. Med. 328(5):313-318 (Feb. 4, 1993)).

Cholesterol is used by the body in the synthesis of the steroid hormones by certain endocrine glands and of bile acids by hepatocytes, and is an essential constituent of cell membranes. It is found only in animals. Related sterols occur in plants, but plant sterols are not absorbed from the gastrointestinal tract. Most of the dietary cholesterol is contained in egg yolks and animal fat.

Cholesterol that is taken up in the intestine is derived directly from the diet and from cholesterol-containing bile salt and acids and free cholesterol synthesized in the liver and secreted into the intestine via bile ducts. Cholesterol esters from the bile and diet are absorbed from the lumen of the small intestine by the intestinal epithelial lining cells and incorporated intracellularly into chylomicrons and, in minor amounts, incorporated into very low density lipoproteins (VLDLs), both of which are secreted into lymphatics that ultimately join the bloodstream. The chylomicrons and VLDLs deliver their triacylglycerols and some of their cholesterol to cells in endothelial, muscle, and adipose tissue. The cholesterol-enriched chylomicron remnants and VLDLs then deliver cholesterol back to the hepatocytes and to other cells of the vascular wall along the way (Ganong, Review of Medical Physiology 249-250 (Lange Medical Publications, 1985). The VLDLs from intestinal and liver cells can be converted to low density lipoproteins (LDLs) by discharge of their triacylglycdrols. LDLs comprise three-fourths of the total plasma cholesterol.

In hypercholesterolemia, the increase in the blood cholesterol level is associated mainly with a rise in LDL concentrations. However, the specific causes of hypercholesterolemia are complicated and varied. At least one kind of hypercholesterolemia is caused by a mutation in the gene for the LDL receptor that moves cholesterol out of the blood, primarily in the liver. Much more commonly, hypercholesterolemia has been associated with high dietary cholesterol, resulting in high cholesterol uptake from the intestine into the circulating blood.

Reduction of hypercholesterolemia results in a delayed onset of atherosclerosis and a decrease in progression of atherosclerosis, thus reducing the risk of coronary heart disease in humans and other primates. Specifically, there is evidence in animals, most notably primates, that relatively complicated plaques induced by hyperlipidemia will regress, and that further progression of atherosclerosis will cease when hyperlipidemia is removed. Therefore, efforts to prevent atherogenesis, to interrupt progression, and perhaps to promote regression of existing lesions by risk factor reduction are warranted (Bierman, “Disorders of the Vascular System: Atherosclerosis and Other Forms of Arteriosclerosis,” in Harrison's Principles of Internal Medicine 1014-1024, (Braunwald et al., 1987)).

Some forms of hyperlipidemia, including hypercholesterolemia, are potentially partially reversible with current techniques of preventive management. However, none of the current techniques is completely successful and many are associated with unwanted side effects and complications. Taking cholesterol-lowering drugs can result in a twenty percent reduction in serum cholesterol. However, drugs are not always warranted for hypercholesterolemia, and some of the hypolipemic drugs, such as Lovastatin, mevastatin, cholestyramine (Questran), Clofibrate, Probucol, and nicotinic acid, may have serious side effects, including an increase in mortality through liver complications, or less severe side effects, such as constipation (cholestyramine), skin flushes, and muscle dysfunction or may have an effect in lowering blood triacylglycerol but not cholesterol. Dietary therapy is usually recommended for all patients with hypercholesterolemia but is not always effective.

It is highly desirable to identify and develop compounds and therapeutic agents which are useful for reducing cholesterol transport from the gut- to the blood or lymph and for the regulation and treatment of cardiovascular disorders (such as high LDL or serum cholesterol levels), obesity, diabetes, elevated body-weight index and other disorders relating to lipid metabolism. It is especially desirable to develop compounds and therapeutic agents that have fewer or less severe side effects than the currently used agents.

### SUMMARY OF THE INVENTION

The present invention is directed to a method for the lowering of levels of LDL cholesterol in an individual comprising administering to the individual an agent which modulates the activity of the protein annexin 2, cyclophilin A, cyclophilin 40, or HSP 56 or the complex of annexin 2 and caveolin I, in the intestinal cells of the individual.

The present invention is further directed to a method for reducing cholesterol transport from the gut into the blood or lymph comprising administering a modulator of the protein annexin 2 or the complex of annexin 2 and caveolin I. In a preferred embodiment, the modulator is an inhibitor of activity of the protein annexin 2 or the complex of annexin 2 and caveolin I. Preferably, the modulator is administered orally.

The present invention is also directed to a method for screening drug candidates for lowering serum LDL levels or for reducing cholesterol transport from the gut into the blood or lymph and includes the steps of screening compounds for the effect of modulating activity of annexin 2 or the complex of annexin 2 and caveolin I. In a preferred embodiment, the modulator is an inhibitor of protein activity or complex binding. Successful drug candidates may optionally be further modified by combinatorial chemistry to generate preferred therapeutic agents.

Compositions of the invention include compounds which are useful for reducing cholesterol transport from the gut to the blood or lymph and for the regulation and treatment of cardiovascular disorders (such as high LDL or serum cholesterol levels), obesity, diabetes, elevated body-weight index and other disorders relating to lipid metabolism which are identified using the screening assays of the invention.

### BRIEF DESCRIPTION OF THE DRAWINGS

FIGS. 1A and B demonstrate alignments of the predicted amino acid sequences of human and zebrafish proteins. FIG. 1A shows human ANX2 versus zebrafish ANX2b. Sequence similarity is 72%. FIG. 1B shows human CAV1 versus zebrafish CAV1. Sequence similarity is 82%.

FIGS. 2A-E demonstrate expression of CAV1 and ANX2. FIG. 2A shows a comparison of human and zebrafish chromosomal segments and reveals synteny between cav1 and anx2 orthologues. FIG. 2B shows expression of cav1 and anx2b in zebrafish larvae. Embryos were fixed in 4% paraformaldehyde and probed with digoxigenin-labeled antisense RNA as described in (22). Top, lateral views of embryos probed for anx2b at 48 hpf (left) and 96 hpf (right). Note strong expression in the epithelium. Scale bar: 500 μm. Bottom, lateral views of embryos probed for cav1 at 48 hpf (left) and 96 hpf (right). Expression is concentrated in the intestinal epithelium, but cav1 can also be seen in the somite boundaries at 48 hpf (left, arrowhead) and in the heart ventricle (96 hpf). Scale bar: 500 μm. FIG. 2C shows identification of a CAV-ANX2b heterocomplex. Equal amounts of protein (20 μg) isolated from adult fish or adult fish intestine were resolved by SDS-PAGE and immunoblotted with ANX2 IgG or CAV1 IgG. The data are representative of 5 independent experiments. FIG. 2D shows equal amounts of protein (20 μg) isolated from the aorta or intestine of C57BL/6 mice that were resolved by SDS-PAGE and immunoblotted with ANX2 IgG or CAV1 IgG. The data are representative of three independent experiments. FIG. 2E shows the approximately 55 kDa band immunoprecipitated from adult intestine using CAV1 IgG as described previously (9) and resolved by SDS-PAGE. The 55 kDa band was recovered from the gel, digested with trypsin and the resulting fragments resolved by SDS-PAGE and transferred to nylon membrane. Five of the fragments were sequenced by mass spectrometry. The sequence of each fragment is shown along with the region to which they correspond in CAV1 or ANX2b. The letter “X” signifies an unidentified amino acid residue.

FIGS. 3A-C demonstrate formation of the CAV1-ANX2b heterocomplex. FIG. 1A shows the effect of cav1 and anx morpholinos on the formation of the CAV1-ANX2b heterocomplex. Embryos (1-8 cell stage) were injected with the following morpholinos: 1) uninjected, 2) cav1, 3) anx2b synthesis 1, 4) anx2b synthesis 2, 5) anx2b mismatched, 6) anx2a. 3T3 cell lysate (20 μg) is loaded directly onto the gel as a positive control for ANX2 and CAV1 (Lane 7). The embryos were then allowed to develop for 48 h. Larvae were processed to generate lysates (approximately 20 embryos/sample) and 50 μg of protein were used for immunoprecipitation with CAV1 IgG or ANX2 IgG as indicated. The precipitates were resolved by SDS-PAGE and immunoblotted with ANX2 IgG or caveolin IgG as indicated. The data are representative of 3-4 independent experiments. FIG. 2B shows rescue of complex formation by anx2b mRNA. Embryos (1-8 cell stage) were injected with anx2b MO (“no RNA” lane) or anx2b MO plus the indicated capped mRNA (control=uninjected). The embryos were allowed to develop for 48 hours. Larvae were processed to generate lysates (approximately 20 embryos/sample) and 50 μg of protein were used for immunoprecipitation with CAV1 IgG (top) or ANX2 IgG (bottom). The precipitates were resolved by SDS-PAGE and immunoblotted with the same IgG used for the precipitation. FIG. 2C shows reformation of the ANX2b-CAV1 complex in vitro. Embryos (1-8 cell stage) were injected with either cav1 or anx2b MO or uninjected (control) and allowed to develop for 48 hours. Lysates were prepared from each class of embryo and immunoprecipitations performed as in (B). For the last lane, lysates from cav1 MO-injected and anx2b MO-injected embryos were mixed together and incubated at room temperature prior to immunoprecipitation. SDS-PAGE and immunoblotting is as in (B).

FIG. 4 shows uninjected and cav1 MO injected embryos fixed at 24 hpf and subjected to whole-mount in situ hybridization using a antisense riboprobe to myoD, a known marker for somitic mesoderm. Embryos are shown in lateral view, anterior to the right. Scale bar: 250 μm.

FIGS. 5A-C demonstrates the effect of reducing ANX2b protein in zebrafish larvae. Newly fertilized embryos (1-8 cell stage) were injected with anx2b MO and allowed to develop. FIG. 5A shows larvae (5 dpf) fed NBD-cholesterol as described (1) then photographed. Uninjected larvae concentrate NBD-cholesterol in the gall bladder (arrowhead) and intestine (arrow). FIG. 5B shows an immunoprecipitation and immunoblot to determine the persistence of anx2b morpholino effect. Embryos were injected with anx2b MO, collected, lysed, immunoprecipitated and immunoblotted as described in legends to FIG. 2. Uninjected control embryos are 48 hpf. For the 120 hour sample, embryos were fed NBD-cholesterol (1) and sorted into low- and high-intestinal fluorescence groups prior to lysis and immunoprecipitation Data are representative of 3-5 experiments with 20-30 larvae/group. Scale bar: 500 μm. FIG. 5C shows the effect of anx2b morpholino on lipid composition. Embryos were injected with anx2b MO, allowed to develop 72 hours, then collected and the total lipid collected and the amount of cholesterol, cholesteryl ester, and triglycerides determined for injected (white bars) and control uninjected (black bars) embryos. Each bar represents the mean of six measurements, 20 embryos per measurement. Differences between injected and control embryos for both cholesterol and cholesteryl ester are statistically significant, p<0.05.

### DETAILED DESCRIPTION OF THE INVENTION

The zebrafish is a striped 2-inch long fish from the Ganges River. As a model system zebrafish provide significant advantages including external development and fertilization, optical clarity of the embryo, and ease of manipulation. In addition, its high fecundity (usually a few hundred but as many as 1000 eggs), short generation time, i.e., time from fertilization to gastrulation is only about 5 hours at 28° C.; somites form between 10-20 hours; and by 24 hours post-fertilization, a recognizable animal with rudimentary eyes and brain is formed. Also ease of mutagenesis and the ability to store large numbers of fish in a relatively small area strengthen its genetic potential. A number of mutations have already been identified from zebrafish and the mutant genes have been cloned. Several of the resulting genes have been homologues of human disease genes. For example, fish model systems now exist for such diseases as sideroblastic anemia [Brownlie, A., et al., Nat genet, 20:244-250, 1998].

To identify genes that regulate cholesterol processing, we undertook a reverse genetic approach by disrupting putative lipid processing genes in zebrafish larvae using morpholino (MO) based antisense oligonucleotides. Using both targeted MO injections and immunoprecipitation experiments coupled with mass spectroscopy analysis, we have determined that Annexin2b (ANX2b), the zebrafish homologue of human annexin2 (ANX2) complexes with Caveolin 1 (CAV 1). MOs directed against either anx2b or cav1 prevent expression of the targeted gene and thus block formation of the protein heterocomplex. MOs directed against anx2b, a gene expressed exclusively in the intestinal epithelium profoundly reduces the ability of larvae to process a fluorescent cholesterol reporter. These experiments provide in vivo data establishing a functional role for ANX2 and validate the zebrafish as a model for identifying new potential drug targets.

The first and potentially most important strategy described here is based on the fact that if the complex of caveolin I and annexin 2 is necessary for the transport of cholesterol from the intestines into the blood stream, blocking the action of annexin 2 or the complex of the two proteins, or the formation of the complex, in the cells of the intestinal wall from performing that transport activity results in decreased transport of cholesterol into the serum. Cholesterol normally enters the intestinal lumen from two sources, food eaten by the individual and from cholesterol excreted from the liver into the bile. If cholesterol transport is inhibited in the intestinal wall cells using an inhibitor of the present invention, serum cholesterol levels will go down, since the cholesterol secreted by the liver will not be re-directed into the blood stream. On the other hand, if the inhibition of cholesterol uptake is selectively performed only in the cells of the intestinal wall, there should be no effect on the levels of HDL in the individual's serum, since the normal transport of cholesterol out of cholesterol producing cells will not be affected. Since the site of annexin 2 or the complex of the two proteins activity that is to be blocked is in the cells of the intestinal wall, it is envisioned that the most convenient mode of delivery of the blocker will be by oral delivery. It is envisioned that the transport activity of the complex can be inhibited in many ways.

One method would be to inhibit the expression of endogenous annexin 2 proteins to reduce the abundance of the proteins. An example of the implementation of this method would be an antisense construct for the annexin 2 gene delivered (either in free form or by liposome or viral vector) through the intestinal tract to the intestinal wall cells. Another method would be to inhibit the activity of the protein by introducing a chemical inhibitor of the activity of the protein or the complex or complex formation. An example of the second method would be the use of an antibody against the protein. In either case, the delivery methodology should be capable of delivering the inhibiting agent to the cells of the intestinal lining.

For the modulation of the protein activity or complex formation using genetic techniques, it is necessary to introduce the genetic elements into the cells of the intestinal epithelium. This can be done by using liposomes of viral vectors carrying the genetic elements orally. Such liposomes or viral vectors can achieve transfection of foreign genetic constructs into the somatic cells with which they come in contact at some frequency dependent on the efficiency of the particular vector. There are several methods that can be used to inhibit gene activity, but amongst those the best known is based on the use of an antisense RNA construct. A genetic construct can be made which encodes the coding region or at least a portion of the coding region of the native annexin 2 gene, in the antisense direction. When such a construct is expressed in cells, the antisense RNA produced interferes with normal gene expression activity in the cells and the native levels of the targeted protein drop. Such an antisense technique can be used to selectively target unwanted cholesterol transport activity in the intestinal lining without interfering with desired protein activity throughout the rest of the body. The known sequence of the human annexin 2 gene is used to enable the implementation of this strategy [Accession number NM_{—}004039]. RNAi may also be used.

RNA interference or “RNAi” is a term initially coined by Fire and co-workers to describe the observation that double-stranded RNA (dsRNA) can block gene expression when it is introduced into worms (Fire et al. (1998) Nature 391, 806-811). dsRNA directs gene-specific, post-transcriptional silencing in many organisms, including vertebrates, and has provided a new tool for studying gene function. RNAi involves mRNA degradation of a target gene. Results showed that RNAi is ATP-dependent yet uncoupled from mRNA translation. That is, protein synthesis is not required for RNAi in vitro. In the RNAi reaction, both strands (sense and antisense) of the dsRNA are processed to small RNA fragments or segments of from about 21 to about 23 nucleotides (nt) in length (RNAs with mobility in sequencing gels that correspond to markers that are 21-23 nt in length, optionally referred to as 21-23 nt RNA). Processing of the dsRNA to the small RNA fragments does not require the targeted mRNA, which demonstrates that the small RNA species is generated by processing of the dsRNA and not as a product of dsRNA-targeted mRNA degradation. The mRNA is cleaved only within the region of identity with the dsRNA. Cleavage occurs at sites 21-23 nucleotides apart, the same interval observed for the dsRNA itself, suggesting that the 21-23 nucleotide fragments from the dsRNA are guiding mRNA cleavage. Isolated RNA molecules (double-stranded; single-stranded) of from about 21 to about 23 nucleotides mediate RNAi. That is, the isolated RNAs mediate degradation of mRNA of a gene to which the mRNA corresponds (mediate degradation of mRNA that is the transcriptional product of the gene, which is also referred to as a target gene). Isolated RNA molecules specific to annexin 2 mRNA, which mediate RNAi, are antagonists useful in the method of the present invention.

Also, with the insight into the intestinal transport function of the caveolin I/annexin 2 complex disclosed here, it becomes possible to screen new drugs for cholesterol lowering function. Chemical entities that will bind with high affinity to the extra-cellular domains of the complex proteins will prove to have cholesterol lowering properties as long as they are capable of passing through the stomach into the intestines without deactivation or digestion.

Another specifically envisioned class of inhibitors of the caveolin I/annexin 2 complex disclosed here includes antibodies, polyclonal or monoclonal, which are directed against the complex and prevent the complex from functioning, e.g., forming or transport. For the approach of using antibodies, it is preferred that the antibodies be raised against the domains of the complex or the complex components which appear to be exposed on the surfaces of those cells.

To make antibodies against these regions, peptides can be prepared that include the amino acid sequences of these regions. These peptides can be used to make polyclonal antibodies by immunizing animals and recovering their serum. Monoclonal antibodies can be made as well. It is also envisaged that antibodies can be made by injecting the peptides into chickens and thus these chickens will produce eggs enriched in the needed antibody as in Yokoyama et al. Am. J. Vet. Res. 54:6:876-872 (1993). The antibodies can be recovered from the egg yolks and prepared separately to expose the antibody to the target.

Screening Assays for Modulators of Caveolin I/Annexin 2 Complex Activity

The invention provides screening assay methods for identifying therapeutic compounds useful for treatments which reduce exogenous cholesterol transport from the gut lumen to the blood or lymph and for the regulation and treatment of cardiovascular disorders (such as high LDL or serum cholesterol levels), obesity, elevated body-weight index and other disorders relating to lipid metabolism which can be used in human patients. The screening assay methods of the invention simplify the evaluation, identification and development of candidate compounds and therapeutic agents for the treatment of such conditions and disorders. In general, the screening methods provide a simplified means for selecting natural product extracts or compounds of interest from a large population, generally a compound library, which are further evaluated and condensed to a few active and selective materials useful for treatments of such conditions and disorders (these treatments are sometimes referred to herein as the “desired purposes of the invention”).

Constituents of this pool are then purified, evaluated, or modified by combinatorial chemistry in order to identify preferred compounds for the desired purposes of the invention.

Compounds that modulate the biological activity of the caveolin I/annexin 2 complex can be identified by their effects on a known biological activity of the complex or each component protein, including but not limited to cellular or microsomal scale assays of efflux of phospholipid, cholesterol or other chemical species, protein level assays of binding specificity, protein stability, regulated catabolism, or its ability to bind proteins, lipids or other factors, expression level or stability of mRNA and precursor RNAs, or, in short, by any activity that identifies a biological effect, characteristic or feature of the complex or each individual protein.

What follows is a general description of a screening assay. More detailed descriptions of certain of these assays are set out in a separate section below.

In one example, drug screening assays are based upon assaying for the ability of the complex to transport cholesterol or another molecule. Zebrafish provide a novel in vivo screening assay. In this model zebrafish (embryo, larvae, and adult fish) can be treated with the potential therapy and then fed NBD-cholesterol. Fluorescent microscopy can then be used to analyze the uptake of cholesterol in the intestine. Biochemical analysis, such as immunoprecipitations and immunoblots, can be performed to examine complex formation in whole fish and intestines as well.

Drug screening assays can be based upon the ability of the caveolin 1 and annexin 2 complex to bind labeled cholesterol in vitro. Using mammalian cells, biochemical analysis, as well as gas chromatography-mass spectrometry analysis, can detect binding of cholesterol to the complex.

Drug screening assays can also be based upon the ability of caveolin I and annexin 2 to interact with each other. Such interaction can be identified by a variety of methods known in the art, including, for example, radioimmunoprecipitation, co-immunoprecipitation, co-purification, and yeast two-hybrid screening. Such interactions can be further assayed by means including but not limited to fluorescence polarization or scintillation proximity methods.

Drug screens can also be based upon functions of the complex deduced upon X-ray crystallography of the complex and comparison of its 3-D structure to that of proteins with known functions.

Drug screens can be based upon a function or feature apparent upon creation of a transgenic or knockout mouse, or upon overexpression or disruption of the complex in mammalian cells in vitro.

Additionally, drug screening assays can also be based upon complex formation deduced upon antisense interference with the gene function. See, Example 1, set forth below. Intracellular localization of the complex, or effects which occur upon a change in intracellular localization of the complex or disruption of the complex, can also be used as an assay for drug screening.

Human and rodent complex can be used as an antigen to raise antibodies, including monoclonal antibodies. Such antibodies will be useful for a wide variety of purposes, including but not limited to fractional studies and the development of drug screening assays and diagnostics. Monitoring the influence of agents (e.g., drugs, compounds) on the expression or biological activity of the complex can be applied not only in basic drug screening, but also in clinical trials. For example, the effectiveness of an agent determined by a screening assay as described herein to modulate annexin 2 gene expression, protein levels, complex formation with caveolin 1 or biological activity can be monitored in clinical trials of subjects exhibiting decreased altered gene expression, protein levels, or biological activity. In addition, serum cholesterol levels can be monitored in subjects.

In such clinical trials, the expression or activity of the complex or each of its components can be used to ascertain the effectiveness of a particular drug.

For example, and not by way of limitation, genes that are modulated in cells by treatment with an agent (e.g., compound, drug or small molecule) that modulates complex biological activity (e.g., identified in a screening assay as described herein) can be identified. Thus, to study the effect of agents on reducing cholesterol transport from the gut to the blood or lymph, or for reducing LDL or serum cholesterol levels, for example, in a clinical trial, cells can be isolated and RNA prepared and analyzed for the levels of expression of caveolin I and/or annexin 2 and other genes implicated in the disorder. The levels of gene expression can be quantified by Northern blot analysis or RT-PCR, or, alternatively, by measuring the amount of protein produced, by one of a number of methods known in the art, or by measuring the levels of biological activity of the complex. In this way, the gene expression can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of caveolin I and/or annexin 2 protein, complex, mRNA, or genomic DNA in the preadministration sample; (iii) obtaining one or more post-administration samples from the subject; (iv) detecting the level of expression or activity of the caveolin I and/or annexin 2 protein, complex, mRNA, or genomic DNA in the post-administration samples; (v) comparing the level of expression or activity of the caveolin I and/or annexin 2 protein, complex, mRNA, or genomic DNA in the pre-administration sample with the caveolin I and/or annexin 2 protein, complex, mRNA, or genomic DNA in the post administration sample or samples; and (vi) altering the administration of the agent to the subject accordingly.

Assays of complex activity include interaction with HDL particles or constituents; interaction with other proteins which facilitate interaction with HDL or its constituents; and measurement of cholesterol efflux.

Alternatively, the effect of candidate modulators on expression or activity may be measured at the level of caveolin I and/or annexin 2 protein production using the same general approach in combination with standard immunological detection techniques, such as Western blotting or immunoprecipitation with an caveolin I and/or annexin 2 protein-specific antibody. Again, useful modulators are identified as those which produce a change in caveolin I and/or annexin 2 protein production.

Agonists, antagonists, or mimetics found to be effective at modulating the level of cellular caveolin I and/or annexin 2 protein expression or activity may be confirmed as useful in animal models (for example, zebrafish, mice, pigs, rabbits, or chickens).

A compound that inhibits annexin 2 (or the complex with caveolin I thereof) protein expression or activity is considered particularly useful in the invention; such a molecule may be used, for example, as a therapeutic to reduce cholesterol transport from the gut to the blood or lymph, or reduce LDL or serum cholesterol levels in an animal (for example, a human).

The Cholesterol Efflux Assay as a Drug Screen

A cholesterol efflux assay measures the ability of cells to transfer cholesterol to an extracellular acceptor molecule. A standard cholesterol efflux assay is set out in Marcil et al., Arterioscler. Thromb. Vasc. BW 19:159-169, 1999, incorporated by reference herein for all purposes.

In this procedure, cells are loaded with radiolabeled cholesterol by any of several biochemical pathways. Cholesterol efflux of cells is measured after incubation for various times (typically 0 to 24 hours) in the presence of HDL3 or purified ApoAl. Cholesterol efflux is determined as the percentage of total cholesterol in the culture medium after various times of incubation. Decreased levels of complex biological activity are associated with decreased cholesterol efflux.

This assay can be readily adapted to the format used for drug screening, which may consist of a multi-well (e.g., 96-well) format. Modification of the assay to optimize it for drug screening would include scaling down and streamlining the procedure, modifying the labeling method, using a different cholesterol acceptor, altering the incubation time, and changing the method of calculating cholesterol efflux. In all these cases, the cholesterol efflux assay remains conceptually the same, though experimental modifications may be made.

Protein-Based Assays

Caveolin I and/or annexin IIb proteins (purified or unpurified) can be used in an assay to determine the ability to form a complex. The effect of a compound on that complex formation is then determined.

Protein Interaction Assays

The caveolin I/annexin 2 complex proteins (either individually or as a complex) are harvested from a suitable source (e.g., from a prokaryotic expression system, eukaryotic cells, a cell-free system, or by immunoprecipitation from cells expressing the protein). The complex is then bound to a suitable support (e.g., nitrocellulose or an antibody). Binding to the support is preferably done under conditions that the complex allow proteins to remain associated. Such conditions may include use of buffers that minimize interference with protein-protein interactions. Complex binding is then tested in the presence and absence of compounds being tested for their ability to interfere with interactions between the members of the complex. Members of the complex may be labeled using, for example, a fluorescent label, to measure the effect of the test compound on complex binding.

Compounds of the Invention

In general, novel compounds and therapeutic agents for reducing cholesterol transport from the gut lumen to the blood or lymph and for the regulation and treatment of cardiovascular disorders (such as high LDL or serum cholesterol levels), obesity, elevated body-weight index and other disorders relating to lipid metabolism are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, NIA). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

Typically, a screening assay, such as a high throughput screening assay, will identify several or even many compounds which modulate the activity of the assay protein. The compound identified by the screening assay may be further modified before it is used in humans as the therapeutic agent. Typically, combinatorial chemistry is performed on the modulator, to identify possible variants that have improved absorption, biodistribution, metabolism and/or excretion, or other important therapeutic aspects. The essential invariant is that the improved compounds share a particular active group or groups which are necessary for the desired modulation of the target protein. Many combinatorial chemistry techniques are well known in the art. Each one adds or deletes one or more constituent moieties of the compound to generate a modified analog, which analog is again assayed to identify compounds of the invention. Thus, as used in this invention, therapeutic compounds identified using a screening assay of the invention include actual compounds so identified, and any analogs or combinatorial modifications made to a compound which is so identified which are useful for treatment of the disorders claimed herein.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their abilities in reducing cholesterol transport from the gut lumen to the blood or lymph and for the regulation and treatment of cardiovascular disorders (such as high LDL or serum cholesterol levels), obesity, elevated body-weight index and other disorders relating to lipid metabolism should be employed whenever possible.

When a crude extract is found to be capable of reducing cholesterol transport from the gut lumen to the blood or lymph and for the regulation and treatment of cardiovascular disorders (such as high LDL or serum cholesterol levels), obesity, elevated body-weight index and other disorders relating to lipid metabolism, further fractionation of the positive lead extract is necessary to isolate the chemical constituent responsible for the observed effect.

Thus, the goal of the extraction, fractionation, and purification process is the careful characterization and identification of a chemical entity within the crude extract having these desired activities. The same in vivo and in vitro assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic value are subsequently analyzed using any standard animal model for the desired disease or condition known in the art.

Because one of the objects of the invention is to inhibit cholesterol transport in the gut but to not inhibit the assembly of HDL particles in peripheral tissues, certain features of preferred compositions of the invention can be identified. In particular, compositions which act locally in the gut or intestinal wall, but which do not circulate widely in the body are preferred. This object may be achieved with compounds which either are incapable of being transported by the blood or lymph or other extra-cellular fluid or particle. This object may also be achieved by obtaining compounds with limited in vivo stability (i.e. short half life upon oral administration) or which are subject to rapid metabolism to inert analogs after absorption by the intestinal wall.

Compositions of the invention, including but not limited to compounds that modulate biological activity or expression of the complex or its components identified using any of the methods disclosed herein, or any preferred analogs of such compositions, may be administered with a pharmaceutically-acceptable diluent, carrier, or excipient, in unit dosage form.

Conventional pharmaceutical practice may be employed to provide suitable formulations or compositions to administer such compositions to patients. Although oral administration is preferred, any appropriate route of administration may be employed, for example, intravenous, perenteral, subcutaneous, intramuscular, intracranial, intraorbital, ophthalmic, intraventricular, intracapsular, intraspinal, intracisternal, intraperitoneal, intranasal, or aerosol administration. Therapeutic formulations may be in the form of liquid solutions or suspension; for oral administration, formulations may be in the form of tablets or capsules; and for intranasal formulations, in the form of powders, nasal drops, or aerosols.

Methods well known in the art for making formulations are found in, for example, Remington: The Science and Practice of Pharmacy, (I 9th ed.) ed. A. R. Gennaro A R., 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel.

A preferred embodiment for use of the compositions of the invention is for combination therapy employing a therapeutic agent of the invention which modulates or inhibits the complex or its component in combination, simultaneous or sequential, with another agent which inhibits endogenous cholesterol synthesis, such as but not limited to a “statin” or HMGCoA reductase inhibitor, etc. This combination therapy is preferred in instances where inhibition of both exogenous cholesterol uptake from the gut and inhibition of endogenous cholesterol synthesis are desired. Therapeutic agents employed in this combination therapy are preferably oral compounds.

### EXAMPLE

Methods

Identification of anx2 and cav Clones

Zebrafish anx clones were previously identified (16). Potential zebrafish cav1 clones were identified by BLAST searching the Zebrafish EST database (www.genetics.wustl.edu/fishlab/frank/cgi-bin/fish/). A single complete cav1 gene was found in IMAGE Consortium clone 3719638, except for the first two codons and the 5′ UTR which were reconstructed from Danio genomic DNA (GenBank accession number AC087254.2).

Morpholino Oligonucleotides

All morpholino oligonucleotides were commercially obtained (GeneTools LLC, Philomath, Oreg.). Sequences used were: cav1, 5′-TGTCCCGTCCTTGTATCCGCTAGTC-3′ (SEQ ID NO: 10); anx2b, 5′-GCCATTTTCCTTAGTTGTTGTAGAG-3′ (SEQ ID NO: 11); anx2b (4 base mismatch), 5′-GCCAATT TCGTTAGTAGTTGAAGAG-3 (SEQ ID NO: 12)′; anx2a, 5′-CTAAGAACTCAGAGACCAAAGCCAT-3′ (SEQ ID NO: 13). MOs were dissolved in Danieau buffer (3) at a concentration of (1-3 μg/μl) and pressure injected into the yolk of early stage larvae (1-8 cell stage) as described (3).

Capping mRNA

To allow transcription of capped mRNA, the anx2a and anx2b open reading frames were separately subcloned into the vector pT3TS and the identity of the insert confirmed by restriction digest analysis. The expression vector was linearized and capped mRNA transcribed using the mMesage mMachine T3 transcription kit (Ambion, Inc., Austin, Tex.) according to the manufacturer's instructions. Product capped mRNA was purified on a CentriSep column (Princeton Separations, Adelphia, N.J.) and aliquots run on agarose gels and subject to spectrophotometry to verify its purity and concentration.

Mapping and Syntenic Analysis

Zebrafish anxs and cav1 where mapped using the LN54 Radiation Hybrid Panel as previously described (19). Syntenic relationships between zebrafish and human genomes were determined by examining mapped zebrafish genes flanking a particular zebrafish gene (maps used were; Mother of Pearl Meiotic Map, GAT Meiotic Map, LN54 Radiation Hybrid Map, for map data see http://mgchd1.nichd.nih.gov: 8000/zfrh/beta.cgi). The protein sequences of the flanking genes were used to identify human orthologues by BLAST searches. The chromosomal locations of human orthologues were determined by searching either the Human Gene Map99 Project or the Human Gene Database.

RNA In Situ Hybridization

Methods for breeding and raising zebrafish were followed as described (21). Embryos were obtained from natural matings of wild-type (Oregon, AB) fish and staged according to criteria previously outlined (22) and by hours post-fertilization (hpf). Digoxigenin-labeled RNA probes synthesized for each gene were hybridized to embryos or larvae at various developmental stages. In situ hybridization for anx2b and cav1 expression was carried out as previously described (22).

SDS-PAGE Analysis

The embryos were lysed in 1 ml of buffer (150 mM NaCl, 1.0% NP-40, 0.5% deoxycholate, 0.1% SDS, 50 mM Tris, pH 8.0) and protein amounts determined by Lowry. Equal amounts of lysate (50 ug) were mixed together in the lysis buffer for 1 hour at 37oC. At the end of the incubation protein A-sepharose beads (blocked with lysis buffer and 30 mg/ml BSA) were used to pre-clear the samples. Pre-cleared samples were than incubated for 18 hours at 4° C. with the appropriate antibody (2 ug/sample) before adding blocked, protein A-sepharose beads and incubating an additional 2 hours at 4° C. The beads were collected by centrifugation, washed five times in high salt RIPA buffer (500 mM NaCl). The samples were than analyzed by SDS-PAGE and immunoblot.

Measuring Cholesterol Uptake

Embryos (uninjected or anx2b MO injected) at 5 dpf were placed at ten per tube in 0.5 ml embryo medium (21) containing methyl-D-glucose, 3-O-[glucose-¹⁴C(U)] (American Radiolabeled Chemicals, Inc.) at a concentration of 12.5 μM and incubated at room temperature for 2 hours. Embryos were then chased with 0.5 ml embryo medium with 125 μM unlabeled glucose and incubated 30 minutes, then rinsed three times with chase solution, transferred to fresh tubes, vigorously homogenized with a pestle, and transferred into scintillation fluid for liquid scintillation counting. Three separate trials with 2 or 3 tubes each of uninjected and injected embryos were performed.

Embryos (uninjected or anx2b MO injected) at 5 dpf were labeled with NBD-cholesterol (1,26) and blind-sorted into three groups based on intestinal fluorescence, each assigned a numerical value: 1=no or weak fluorescence, 2=moderate, 3=strong; total n=41 uninjected, 55 injected.

Approximately 80 embryos at 72 hpf were placed in 0.7 ml of MBST/OG (25 mM MES, pH 6.5, 0.15 M NaCl, 1% (v/v) Triton X-100, 60 mM octylglucoside, 0.1% (w/v) SDS) and homogenized with a Tissue-Tearor™ homogenizer (Biospec Products, Inc). The sample was then sonicated (50 watts) for ten seconds, three times on ice, centrifuged at 1000×g for 5 minutes and the supernatant collected. Neutral lipids were isolated by Solid Phase Extraction (SPE) on an Oasis HLB 5 cc/200 mg LP Glass Cartridges (Waters, Milford Mass.). The columns were pre-conditioned with 3 ml of methyl tetr-butyl ether, then 3 ml methanol, and finally 3 ml of water. The sample (0.5 ml) was mixed with acetic acid (1 ml) and 5α-androstane-3α-17β-diol (recovery standard, 40 μl) and immediately applied to the column. The column was washed with 2 ml of 2% acetic acid followed by 2 ml of methanol (2%):acetic acid (2%). Neutral lipids were eluted with 3 ml of methyl tetr-butyl ether:methanol:acetic acid (75:23:2). The eluted material was dried under nitrogen, dissolved in 150 μl of hexane:isopropanol (3:2) and transferred to a gas chromatography vial. The material was dissolved in 20 μl of hexamethyldisilazane: pentafluoropropionic anhydride:acetonitrile (100:2:20) for 20 minutes at room temperature and 0.2 ml was then injected onto a Chemstation gas chromatograph with a mass selective detector, Model 6890 (Agilent Technologies, Wilmington, Del.) and equipped with a SPE HT5 Aluminum Clad Fused Silica Capillary Column, 12 m, 0.32 mm ID, 0.1 um film (Supelco, Bellefonte, Pa.). The inlet temperature was 220° C., the interface temperature was 340° C. and the oven program was 140° C., hold 3 min, 20° C./min to 380° C., hold 5 min. Method of lipid detection was SIM mode with the following mass numbers: 416 for 5α-androstane-3α-17β-diol, 436 for TMS-cholesterol, 368 for cholesterol esters, 98 for triglycerides. A two step procedure was used to quantify the lipids. The first step included analyzing a test sample containing different lipids to determine the coefficient of recovery (5α-cholest-7en-3β-ol, cholesteryl heptadecanoate, triheptadecanoate, 5α-androstane-3α-17β-diol, cholesterol, estradiol. The second step contained the unknown sample and 5α-androstane-3α-17β-diol.

In Vivo Assay for Cholesterol Transport

Zebrafish larvae were labeled (2 h) with NBD-Cholesterol prepared as previously described (1) in embryo medium (21), anesthetized (tricaine, 170 ug/ml) and placed in depression slides. Fluorescent images were captured using a Zeiss Axiocam 2 mounted on a Leica MZFL-III. For statistical analysis intestinal fluorescence was scored on a three point scale (weak, moderate and bright) and analyzed (ANOVA).

Results

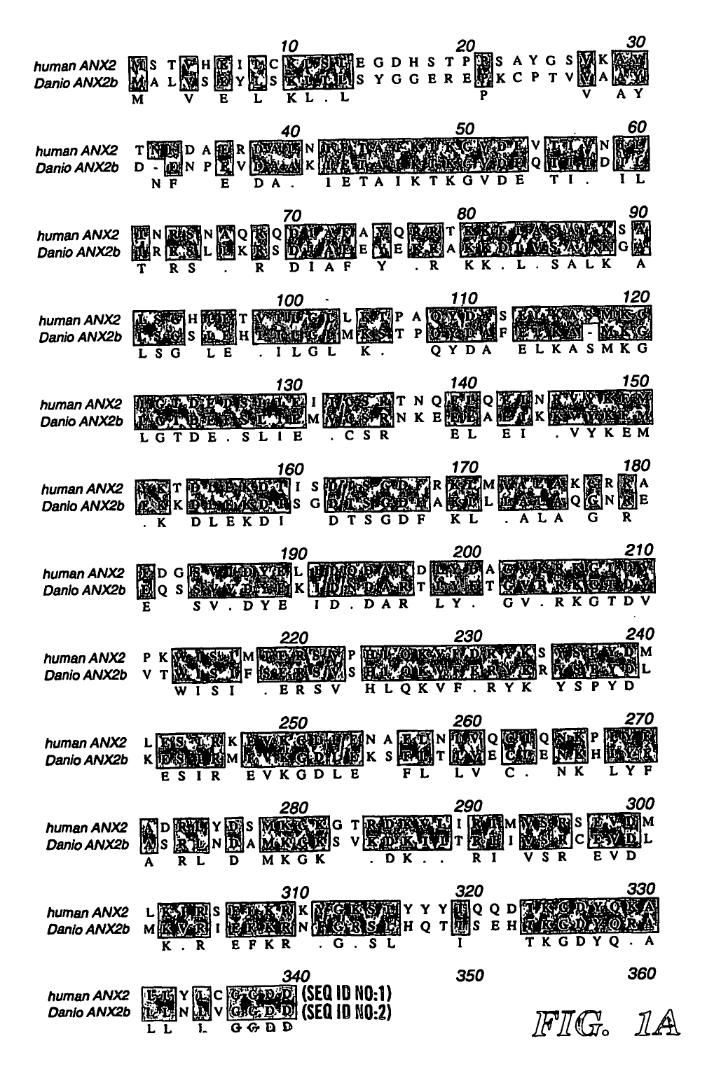

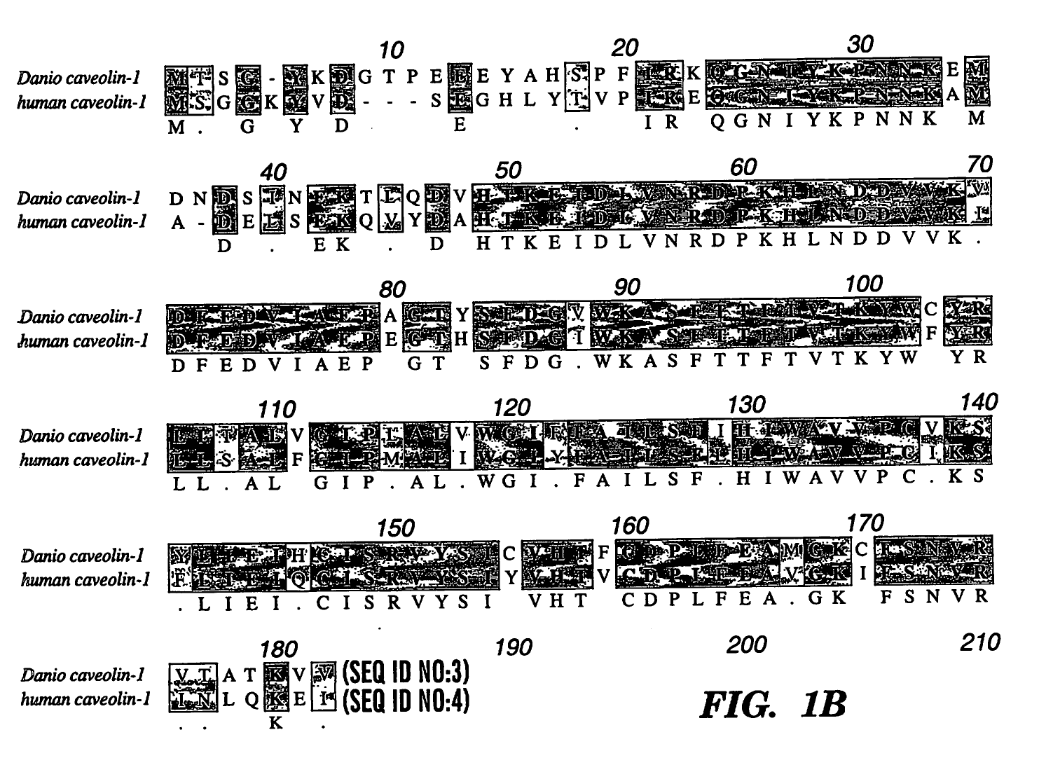

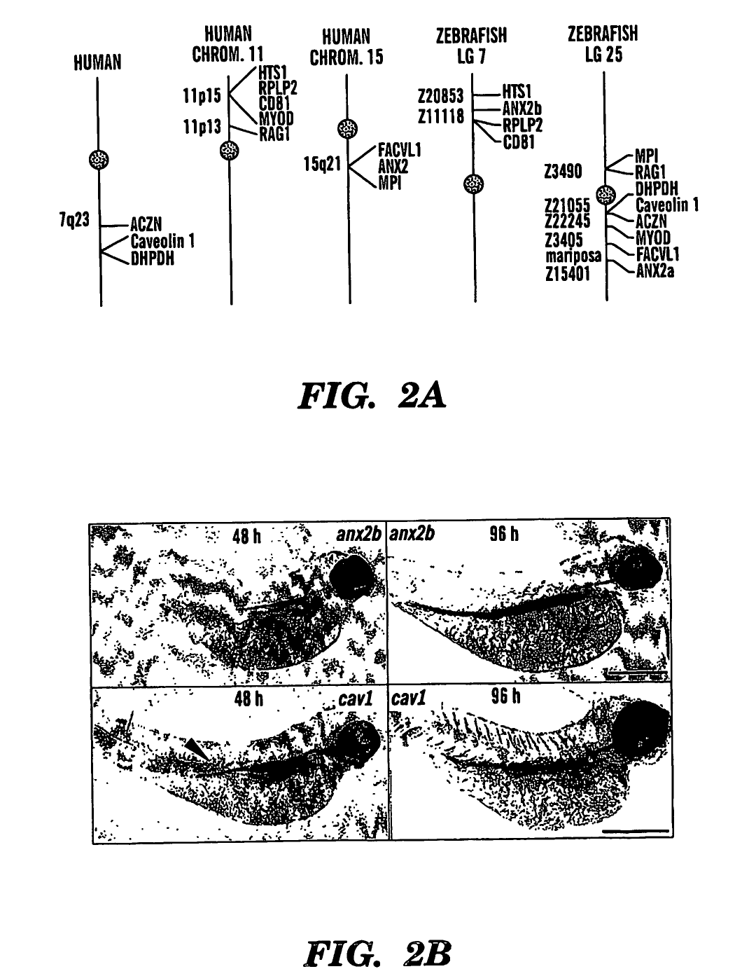

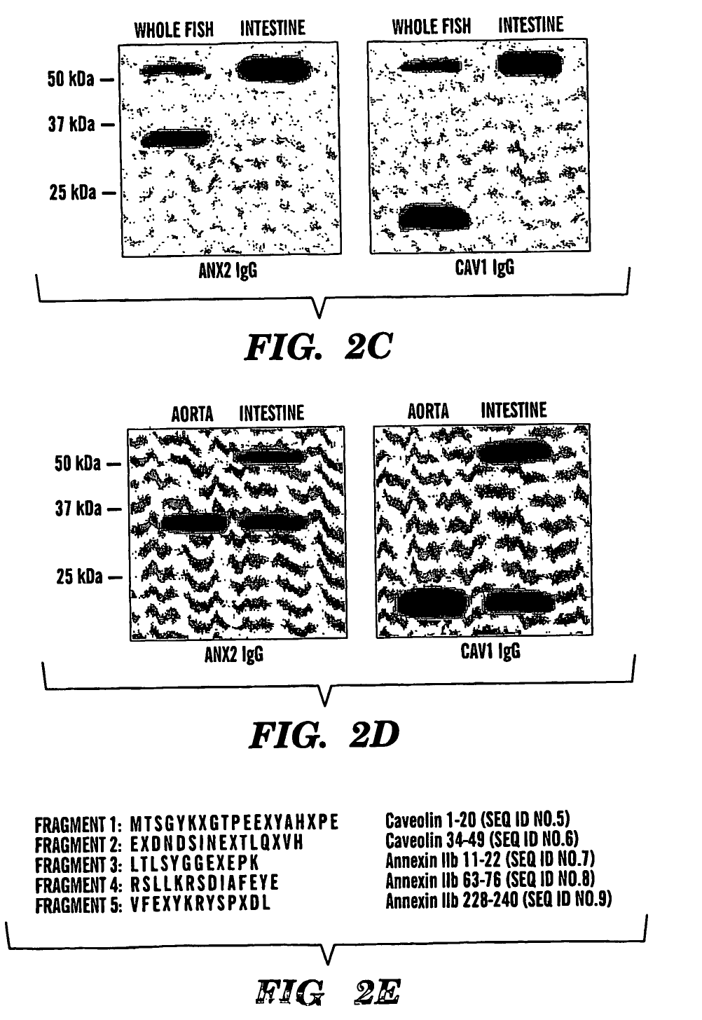

As a first step to study the in vivo function of CAV, we identified and cloned the zebrafish orthologue of mammalian cav1 using EST sequences generated by the Zebrafish Genome Resources Project (http://zfish.wustl.edu). Zebrafish ANX2b and CAV1 (FIG. 1) were highly conserved (72% and 82% similarity to human proteins respectively). To confirm that these genes were orthologous to their human counterparts, the chromosomal position of zebrafish anx2b (16) and cav1 was determined. On the basis of the surrounding mapped genes, we found that these particular chromosomal regions were syntenic with the corresponding human chromosomal segments (FIG. 2A) indicating that we identified the correct orthologues and not related family members.

Expression of cav1 and anx2b transcripts in zebrafish embryos were determined by whole-mount in situ hybridization (FIG. 2B) (23). anx2b was expressed in the intestinal epithelium at both 48 hours post fertilization (hpf) and 96 hpf as was cav1. However, cav1 was also detected in the intersomite spaces at 48 hpf and in the heart ventricle at 96 hpf. The expression of the two genes shows significant colocalization, consistent with our hypothesis of an interaction between ANX2b and CAV1 proteins.

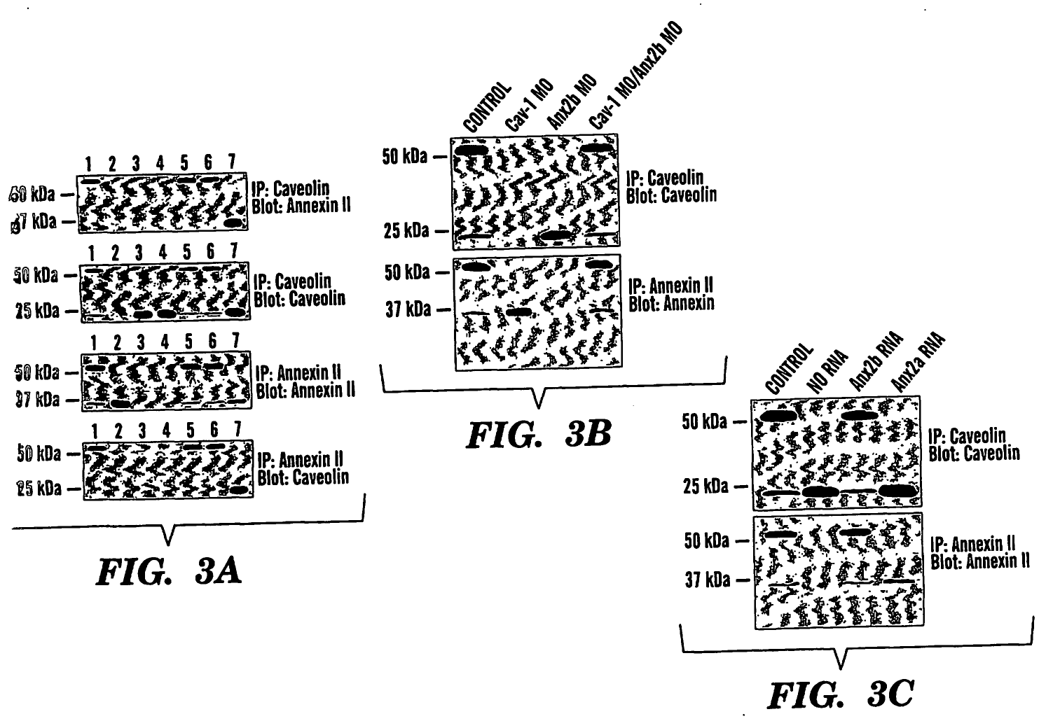

To obtain biochemical evidence of an interaction between ANX2b and CAV1 proteins in vivo, we performed a series of immunoblots on adult whole fish and intestines (FIG. 2C) and on 48 hpf whole embryos (FIG. 3A, lane 1). 60-80% of ANX2b (38 kDa) and CAV1 (21 kDa) proteins are present as uncomplexed monomers in adult whole fish with the remainder visible in a higher molecular weight complex of approximately 55 kDa. In adult fish intestine, essentially all the ANX2b and CAV1 are present in the 55 kDa complex which is consistent with the in situ hybridization data at 48 hpf (a stage where cav1 expression almost entirely overlaps with anx2b). The size of this complex agrees with the predicted molecular weight of a 1:1 heterocomplex of ANX2b and CAV1 (20.6 kDa+38.4 kDa=59 kDa predicted weight).

To confirm the identity of proteins in the 55 kDa band, peptide fragments from the band were subjected to mass spectrometry analysis (FIG. 2E). Five peptide fragments were identified, all of which corresponded to the predicted amino acid sequences of ANX2b and CAV1 proteins, confirming that these proteins form a tightly associated heterocomplex. Further experiments revealed an anomalous peptide fragment that was resistant to Edman degradation. Additional proteolytic digests of the purified anomalous fragment resolved two short peptides whose sequences matched amino acid residues 7-19 of CAV1 and 18-29 of ANX2b, respectively, suggesting their proximity to the linkage site (data not shown). We also tested whether this finding of a heterocomplex could be generalized to mammals by assaying adult mouse aorta and intestine (FIG. 2D). ANX2b and CAV1 do not form a detectable amount of heterocomplex in mouse aorta, whereas approximately 50% of these proteins were detected as a heterocomplex in mouse intestine indicating that this SDS resistant heterocomplex is a general feature likely to be present in all vertebrates.

Next, immunoprecipitations and immunoblots were performed on 48 hpf zebrafish embryos (FIG. 3A; lanes are identical in all four blots). In control embryos, the majority of both ANX2b and CAV1 were present as a heterocomplex, while significant amounts of uncomplexed monomer were also present (FIG. 3A lanes 1). In embryos injected with morpholino oligonucleotide (MO) to block transcription of cav1, CAV1 protein was not detected (FIG. 3A, lane 2) and ANX2b was seen only as a monomer (FIG. 3A, lane 2). Conversely, embryos injected with anx2b MO did not contain ANX2b protein (FIG. 3A, lanes 3 and 4) while CAV1 was only detected as a monomer (FIG. 3A, lanes 3 and 4). Embryos injected with anx2b MO containing 4 bases mismatched (FIG. 3A, lanes 5) or anx2a MO (FIG. 3A, lanes 6) contained the CAV1-ANX2b heterocomplex exactly like the uninjected embryos. In contrast, while in 3T3 cells a CAV1-ANX2 complex exists as previously described (9) (CAV1 immunoprecipitation “pulls down” ANX2; FIG. 3A, lane 7) the heterocomplex is not stable in SDS as evidenced by the appearance of only the monomer. We conclude that the ANX2-CAV 1 heterocomplex observed in adult fish intestine is already present in 48 hpf embryos and that ANX2a is not required for the formation of this complex.

We also examined the ability of the complex to re-form both in vivo and in vitro. Protein extracts of 48 hpf cav1 MO injected embryos show only ANX2 protein monomer and no complex (FIG. 3B, lane 2); conversely, anx2b MO-injected embryos show only CAV protein monomer (FIG. 3B, lane 3). When extracts of anx2b MO injected embryos are incubated with extracts of cav1 MO injected embryos prior to SDS-PAGE, the heterocomplex reforms (FIG. 3B, lane 4). In addition, we determined that co-injecting anx 2b mRNA along with anx2b MO induces transcription of an ANX2b protein that can form a heterocomplex with CAV1 (FIG. 3C, lane 3). In contrast, co-injection of anx2a mRNA with anx2b MO, did not lead to the formation of detectable heterocomplex (FIG. 3C, lane 4).

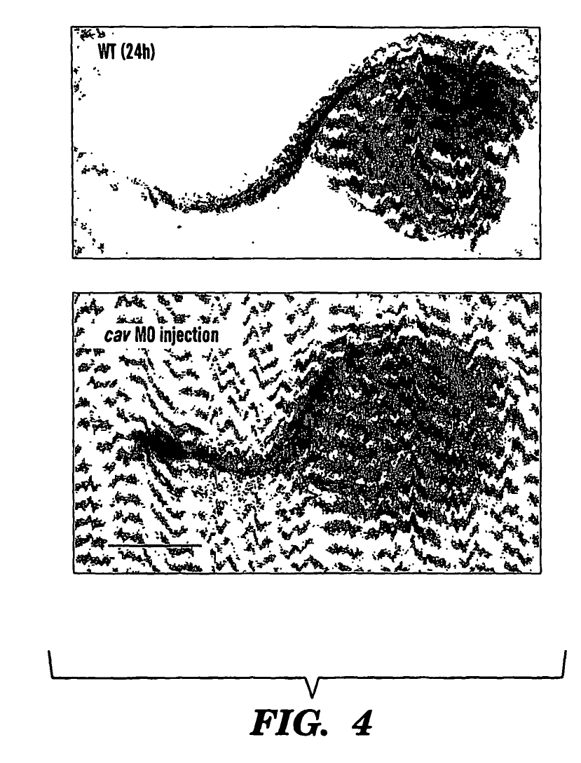

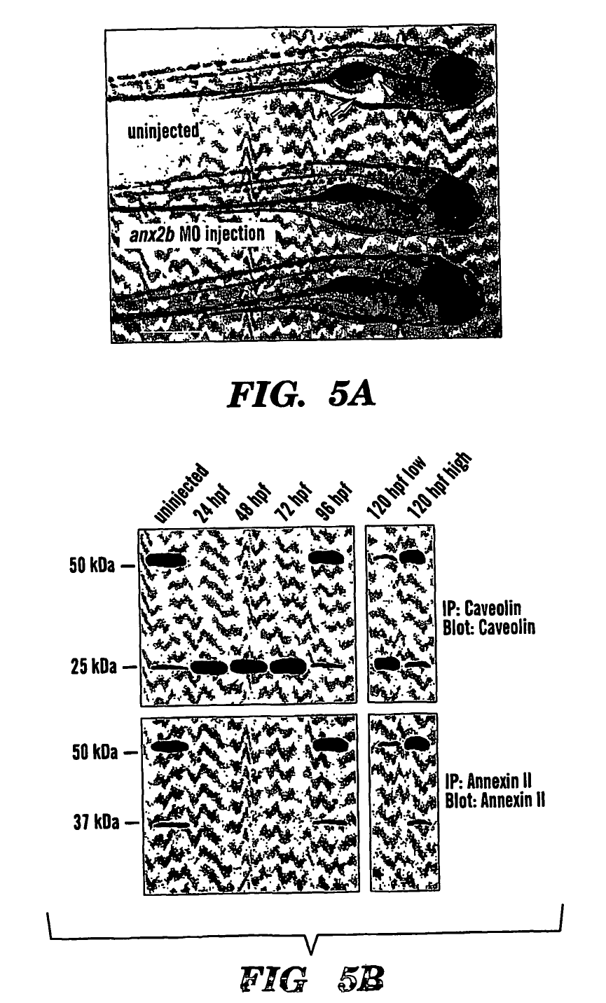

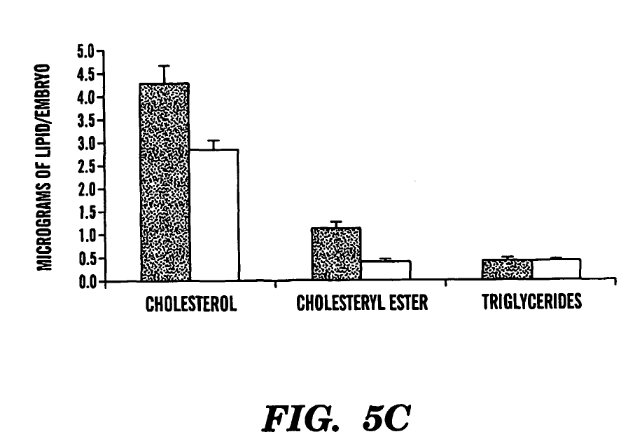

To test the physiologic significance of this complex, embryos were injected with MOs against anx2b and cav1 and larval cholesterol transport was observed as previously described (1). Embryos (5 dpf) injected with anx2b MO did not show any obvious developmental abnormalities, yet a subset of the injected larvae failed to accumulate the fluorescent cholesterol analog in the digestive tract (FIG. 5A). Fluorescent microscopy of live uninjected larvae revealed NBD-cholesterol in the intestine (arrow) and gallbladder (arrowhead). However, an analysis of gut fluorescence in anx2b MO injected larvae from three independent experiments indicated a significant reduction in intestinal fluorescence (ANOVA p<0.00001). Similar experiments using cav1 MO were not possible because injected larvae show early developmental abnormalities including defects in axis elongation and somite patterning (FIG. 4, bottom) and failed to survive to a stage where NBD-cholesterol is ingested. To more closely examine the somite defects embryos (24 hpf) were subjected to whole-mount in situ hybridization using myoD, a known marker for somitic mesoderm, as a riboprobe. The uninjected embryos had well-defined chevron shaped somites and normal elongation of the body axis (FIG. 4, top), in contrast to the abnormal somites of cav1 MO injected larvae.

Additional experiments were performed to determine the duration of the effectiveness of anx2b MO in suppressing expression of its target mRNA. In embryos injected with anx2b MO, no ANX2b and thus no complex is detected at 24, 48, and 72 hpf (FIG. 5B, lanes 2-4). ANX2b is again detected at 96 hpf, with most of the protein present as heterocomplex with CAV1 (FIG. 5B, lane 5). However, when the injected embryos at 120 hpf were fed NBD-cholesterol and sorted into high and low intestinal fluorescence, two distinct populations emerged. Embryos showing poor intestinal fluorescence show very low levels of ANX2b heterocomplex (FIG. 5B, lane 6) while embryos showing a wild-type level of intestinal fluorescence show levels of ANXb2 heterocomplex similar to uninjected embryos (FIG. 5B, lane 7). This experiment further confirms that the amount of ANX2b-containing complex present in the zebrafish intestine is directly related to the ability to efficiently take up exogenous fluorescently-labeled cholesterol.

We also looked at the effect of anx2b MO on the levels of cholesterol, cholesteryl ester, and triglycerides in 72 hpf embryos. As shown in FIG. 5C, anx2b MO injected embryos show significantly lower levels of cholesterol and cholesteryl ester than uninjected embryos, while the level of triglycerides is unaffected by anx2b MO. At this stage of zebrafish embryonic development, all lipids are thought to derive from embryonic yolk rather than exogenous food sources. Our results imply that yolk lipids must be absorbed through the intestinal epithelium; disruption of anx2b specifically alters cholesterol and cholesteryl ester metabolism but does not cause a general defect in lipid uptake. Similarly, the uptake of ¹⁴C-labeled glucose in 5 dpf embryos is the same for uninjected and anx2b MO injected embryos (data not shown), further confirming that disrupting the ANX2b-CAV1 complex has a specific effect on cholesterol uptake and is not causing a general disruption of transport across the intestinal epithelium.

These data demonstrate that CAV 1 and ANX2 form a heterocomplex that mediates intestinal cholesterol uptake. Although previous studies have demonstrated a role for CAV1 and ANX2 in intracellular sterol trafficking (9) this is the first demonstration of a physiological role for CAV 1 and ANX2 in the absorption of exogenous cholesterol. The intestinal CAV-ANX heterocomplex is unique in that it is stable in the presence of detergents and high temperatures. Both the intestinal heterocomplex and the CAV1-ANX2 complex observed in cultured cells also associate with cyclophilin 40 and cyclophilin A (data not shown). In cultured cells either ANX2 or heat shock protein 56 associates with CAV1; ANX2 is needed for the uptake of exogenous cholesterol and heat shock protein 56 is needed for the efflux of newly synthesized cholesterol (9). It is tempting to speculate that the CAV1-ANX2 heterocomplex is tightly associated in the intestine so that the direction of cholesterol movement is shifted towards uptake and not efflux. Importantly, the same tightly associated heterocomplex exists in the intestine of both fish and mice suggesting that this is a general mechanism of intestinal sterol uptake.

The preceding examples and specification are illustrations of the invention which are non-limiting examples of the invention more generally described by the claims below.

### REFERENCES

All references described herein are incorporated herein by reference.
1. S. A. Farber, et al., Science 292, 1385-8. (2001).
2. J. Heasman, Developmental Biology 243, 209-14 (2002).
3. A. Nasevicius, S. C. Ekker, Nat Genet 26, 216-20. (2000).
4. E. J. Smart, et al., Mol Cell Biol 19, 7289-304. (1999).
5. T. V. Kurzchalia, et al., J Cell Biol 118, 1003-14. (1992).
6. K. G. Rothberg, et al., Cell 68, 673-82. (1992).
7. A. Uittenbogaard, Y. Ying, E. J. Smart, J Biol Chem 273, 6525-32. (1998).
8. A. Uittenbogaard, E. J. Smart, J Biol Chem 275, 25595-9. (2000).
9. A. Uittenbogaard, W. V. Everson, S. V. Matveev, E. J. Smart, J Biol Chem 277, 4925-31. (2002).
10. E. J. Smart, Y. Ying, W. C. Donzell, R. G. Anderson, J Biol Chem 271, 29427-35. (1996).
11. F. J. Field, in Intestinal Lipid Metabolism C. M. Mansbach, P. Tso, A. Kuksis, Eds. (Kluwer Academic, New York, 2001) pp. 235-255.
12. C. Mirre, L. Monlauzeur, M. Garcia, M. H. Delgrossi, A. Le Bivic, Am J Physiol 271, C887-94. (1996).
13. F. J. Field, E. Born, S. Murthy, S. N. Mathur, J Lipid Res. 42, 1687-1698 (2001).
14. B. Seaton, Annexins: Molecular Structure to Cellular Function (R. G. Landes Company, Austin, 1996).
15. V. Gerke, S. E. Moss, Physiol Rev 82, 331-71. (2002).
16. S. A. Farber, E. S. Olson, M. E. Halpern, submitted (2002).
17. Omitted
18. Omitted
19. N. Hukriede, et al., Proc Natl Acad Sci USA 96, 9745-50 (1999).
20. Omitted
21. M. Westerfield, The Zebrafish Book (University of Oregon, Eugene, ed. 3rd, 1995).
22. C. Kimmel, W. Ballard, S. Kimmel, B. Ullmann, T. Schilling, Dev Dyn 203, 253-310 (1995).
23. C. Thisse, B. Thisse, T. Schilling, J. Postlethwait, Development 119, 1203-15 (1993)

What is claimed is:

1. A method for inhibiting cholesterol uptake in the gut comprising the step of administering to an individual an inhibitor of annexin 2 or a complex of annexin 2 and caveolin 1.

2. A method for lowering levels of LDL cholesterol in an individual comprising the step of administering to that individual an agent which reduces the activity of annexin 2 or a complex of annexin 2 and caveolin 1.

3. The method of claim 2, wherein the agent acts by disrupting complex formation.

4. A method for identifying drugs that can lower serum cholesterol levels comprising assaying the drug to test if it can disrupt a complex of caveolin I and/or annexin 2.

5. A method for reducing transport of cholesterol from the gut to the blood comprising administering an annexin 2 or a complex of annexin 2 and caveolin 1 modulating compound to an animal and thereby reducing such transport.

6. A screening assay for determining whether a candidate compound is useful for reducing transport of cholesterol from the gut to the blood or lymph, or for lowering LDL or serum cholesterol levels comprising (a) providing an assay system having a measurable biological activity of annexin 2 or a complex of annexin 2 and caveolin 1; (b) contacting the assay with the candidate compound; and (c) measuring biological activity, wherein modulation of biological activity, relative to an assay not contacted with the candidate compound, indicates that the candidate compound is useful for the treatment of said disease or condition.

7. The screening assay of claim 6, wherein the assay system is a cell based system.

8. The screening assay of claim 6, wherein the assay system is a zebrafish based system.

9. The screening assay of claim 6, wherein the assay system is a cell free system.

10. A screening assay for identifying a substance to be tested for an ability to reduce transport of cholesterol from the gut to the blood or lymph, or for lowering LDL or serum cholesterol levels comprising assaying the ability of the substance to modulate expression or activity of the annexin 2 or a complex of annexin 2 and caveolin 1 by
(a) exposing a subject or cell to a test substance;
(b) assaying the expression level of, or the activity of, the annexin 2 or a complex of annexin 2 and caveolin 1 in the subject or cell and a control subject or cell which is not exposed to the test substance; and (c) comparing the expression level of or the activity of the annexin 2 or a complex of annexin 2 and caveolin 1 in the subject or cell to the control subject or cell, wherein a test substance that modulates expression or activity of the annexin 2 or a complex of annexin 2 and caveolin 1 is a substance to be tested for an ability to ameliorate said disease or condition.

11. A compound for reducing transport of cholesterol from the gut to the blood or lymph or for lowering LDL or serum cholesterol levels identified by a screening assay wherein such compound antagonizes the biological activity of annexin 2 or a complex of annexin 2 and caveolin 1.

12. The compound of claim 11, wherein such compound is administered orally.

13. A compound useful for reducing transport of cholesterol from the gut to the blood or lymph, or for lowering LDL or serum cholesterol levels identified by (a) providing an assay system having a measurable biological activity of annexin 2 or a complex of annexin 2 and caveolin 1; (b) contacting the assay with the candidate compound; and (c) measuring biological activity, wherein reduction of biological activity, relative to an assay not contacted with the compound, indicates that the candidate compound is so useful.

14. A screening assay for determining whether a candidate compound is useful for reducing transport of cholesterol from the gut to the blood or lymph, or for lowering LDL or serum cholesterol levels comprising contacting a zebrafish larvae with a labeled cholesterol and a test compound; and measuring the uptake of the labeled cholesterol, wherein a decrease in cholesterol uptake, compared with a control without the test compound, indicates that the candidate compound is useful for the treatment of said disease or conditions.


SEQUENCE LISTING (13 sequences)

SEQ ID NO: 1 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKVGKSLYYYIQQDTKGDYQKALLYLCGGDD

SEQ ID NO: 2 - protein, 337 aa, Danio rerio
MALVSEYLSKLTLSYGGEREPKCPTVVAAYDFNPEVDAAKIETAIKTKGVDEQTIIDILTRRSLLKRSDIAFEYEKRAKKDLVSALKGALSGSLEHLILGLMKSTPQYDAFELKAMKGLGTDEESLIEMVCSRNKEELAEIKKVYKEMFKKDLEKDISGDTSGDFAKLLLALAQGNREEQSSVVDYEKIDNDARTLYETGVRRKGTDVVTWISIFSERSVSHLQKVFERYKRYSPYDLKESIRMEVKGDLEKSFLTLVECLENKHLYFASRLNDAMKGKSVKDKIITRIIVSRCEVDLMKVRIEFKRNFGRSLHQTISEHTKGDYQRALLNLVGGDD

SEQ ID NO: 3 - protein, 181 aa, Danio rerio
MTSGYKDGTPEEEYAHSPFIRKQGNIYKPNNKEMDNDSINEKTLQDVHTKEIDLVNRDPKHLNDDVVKVDFEDVIAEPAGTYSFDGVWKASFTTFTVTKYWCYRLLTALVGIPLALVWGIFFAILSFIHIWAVVPCVKSYLIEIHCISRVYSICVHTFCDPLFEAMGKCFSNVRVTATKVV

SEQ ID NO: 4 - protein, 178 aa, Homo sapiens
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

SEQ ID NO: 5 - protein, 19 aa, Danio rerio
  MOD_RES (7): unidentified amino acid
  MOD_RES (13): unidentified amino acid
  MOD_RES (17): unidentified amino acid
MTSGYKXGTPEEXYAHXPE

SEQ ID NO: 6 - protein, 16 aa, Danio rerio
  MOD_RES (2): unidentified amino acid
  MOD_RES (10): unidentified amino acid
  MOD_RES (14): unidentified amino acid
EXDNDSINEXTLQXVH

SEQ ID NO: 7 - protein, 12 aa, Danio rerio
  MOD_RES (9): unidentified amino acid
LTLSYGGEXEPK

SEQ ID NO: 8 - protein, 14 aa, Danio rerio
RSLLKRSDIAFEYE

SEQ ID NO: 9 - protein, 13 aa, Danio rerio
  MOD_RES (4): unidentified amino acid
  MOD_RES (11): unidentified amino acid
VFEXYKRYSPXDL

SEQ ID NO: 10 - DNA, 25 bp, Artificial Sequence
  Description of Artificial Sequence Synthetic oligonucleotide
tgtcccgtccttgtatccgctagtc

SEQ ID NO: 11 - DNA, 25 bp, Artificial Sequence
  Description of Artificial Sequence Synthetic oligonucleotide
gccattttccttagttgttgtagag

SEQ ID NO: 12 - DNA, 25 bp, Artificial Sequence
  Description of Artificial Sequence Synthetic oligonucleotide
gccaatttcgttagtagttgaagag

SEQ ID NO: 13 - DNA, 25 bp, Artificial Sequence
  Description of Artificial Sequence Synthetic oligonucleotide
ctaagaactcagagaccaaagccat